(19) United States
(12) Patent Application Publication
(10) Pub. No.: US 2020/0254852 A1
(43) Pub. Date: Aug. 13, 2020

(54) AUTOMATIC CAR AIR FRESHENER

(71) Applicant: Rachel Gee, Bullard, TX (US)

(72) Inventor: Rachel Gee, Bullard, TX (US)

(21) Appl. No.: 16/316,183

(22) PCT Filed: Jul. 8, 2016
(86) PCT No.: PCT/US2016/041621

§ 371 Date: Jan. 8, 2019

(51) Int. Cl. B60H 3/00; A61L 9/14; A61L 9/12

(52) U.S. Cl. CPC B60H 3/0021; B60H 3/0035; A61L 9/14; A61L 2209/16; B60H 2003/0042; B60H 2003/0064; A61L 2209/111; A61L 9/12

(57) ABSTRACT

An automatic car air freshener system is disclosed that is configured to integrate into existing ducting of a vehicle and selectively disperse a measured amount of scent matter into the ducts for dispersion into the cabin of the vehicle. The system includes a storage unit containing a volume of scented matter, a release mechanism in communication with the storage unit, and a control unit in communication with the release mechanism and configured to regulate the dispersion of the scented matter from the storage unit in accordance with the desires of a user. The control unit is an interface for the passenger to regulate scent dispersion. A sensor is also optionally included to monitor air quality from the ambient air.

### TECHNICAL FIELD

The present invention relates to a car freshening system and more particularly to a system used to provide a direct injection of scent into the vent system of a vehicle so as to equally disperse such scent into the cabin.

### DESCRIPTION OF THE PRIOR ART

Presently many types of methods are conceived of and produced to regulate or mask the scent of odors in the air we breathe. Odors within confined locations are often more potent to individuals than odors outside. Different methods are used to try and mask such odors. The main masking method is to introduce a more pleasing scent into the environment. The idea is to have the new scent cover over the smell of the unpleasant odor. The difficulty is in making the scent disperse equally within the environment. Some methods choose to disperse potent levels of scent in an effort to ensure there was sufficient coverage. Others involve the use of multiple devices to increase the number of scent emitting locations.

Additionally, most methods appear to let the smells disseminate naturally with the currents of air within the confined environment. Others have used electrical power to force the dissemination. It appears in most all these methods, the user is typically not able to precisely adjust or regulate the amount of smell emitted, the timing of the release of smell, and conveniently change the smells available for dissemination with minimal effort.

An improved car air freshener system is necessary. Although great strides have been made, considerable shortcomings remain.

### SUMMARY OF THE INVENTION

The present application discloses a system that is incorporated into a vehicle for the selected dispersion of a scent in a determined potency, at a particular time, and of a particular desired fragrance or scent. The system of the present application is configured to selectively disperse this scent into the vent system of the vehicle so as to allow the existing temperature regulating system of the vehicle to more evenly and adequately ensure an even distribution into the vehicle compartment.

The system is configured to include a freshener system having a storage unit in communication with a release mechanism for regulating the release of the scent. A control unit is included within an accessible area of the vehicle cabin to permit an individual to manipulate and control the various functions and features of the system. The release mechanism passes the scent through an exit port in communication with the vent ducting of the vehicle. The scent is mixed in with the existing air flows through the vent system of the vehicle and is dispersed through the vehicle's air vent.

The invention may take many embodiments but features the ability to locate the exit port at various locations. For example, the exit port where the scent is introduced into the airflow may occur at any of the following locations: 1) prior to the blower motor of the vehicle wherein the blower motor is used to draw in the scent and subsequently passes it through the vents of the vehicle cabin; 2) directly into a portion of the blower motor where the scent still passes within the blower and then travels into the vehicle compartment; and 3) at a location after the blower motor wherein the blower motor induces a flow of air that pushes the scent into the vehicle compartment. Ideally this third option would occur prior to the ducting being split so as to feed each interior vent. This ensures the ability to more adequately achieve a uniform dissemination of the scent within the vehicle compartment. In this way, this system overcomes the disadvantages inherent in existing methods of distributing a scent.

The more important features of the system have thus been outlined in order that the more detailed description that follows may be better understood and to ensure that the present contribution to the art is appreciated. Additional features of the system will be described hereinafter and will form the subject matter of the claims that follow.

Many objects of the present system will appear from the following description and appended claims, reference being made to the accompanying drawings forming a part of this specification wherein like reference characters designate corresponding parts in the several views.

Before explaining at least one embodiment of the system in detail, it is to be understood that the system is not limited in its application to the details of construction and the arrangements of the components set forth in the following description or illustrated in the drawings. The system is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present system. It is important, therefore, that the claims be regarded as including such equivalent constructions insofar as they do not depart from the spirit and scope of the present system.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a side view of an automatic car air freshener system in a vehicle according to an embodiment of the present application;

FIG. 2 is a first schematic of the automatic car air freshener system of FIG. 1 in relation to parts of the vehicle;

FIG. 3 is a schematic of the automatic car air freshener system of FIG. 1;

FIG. 4 is an exemplary schematic of a control unit in the automatic car air freshener system of FIG. 1;

FIG. 5 is an interior perspective view of the vehicle of FIG. 1 with the automatic car air freshener system.

While the system of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the device described herein may be oriented in any desired direction.

The system in accordance with the present application overcomes one or more of the above-discussed problems commonly associated with existing methods and devices of masking odors and providing desirable scents into the air. Specifically, the system of the present application is configured to operate in particular within a vehicle and provide a passenger the capability of selectively introducing a designated scent into the air. The scent is introduced into the vent system of the vehicle and distributed via the forced air flow via the blower motor. The system is configured to grant the user the ability to adjust the timing of dispersion, potency of dispersion, and the particular scent dispersed in embodiments where multiple storage units are available. Additionally, the control unit is configured to be an interface for the user to regulate the system and can monitor the air quality in the environment and automatically regulate dispersion of the scent. These and other unique features of the system are discussed below and illustrated in the accompanying drawings.

The system will be understood, both as to its structure and operation, from the accompanying drawings, taken in conjunction with the accompanying description. Several embodiments of the assembly are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The system of the present application includes a storage unit containing a volume of scented matter, a release mechanism in communication with the storage unit, and a control unit in communication with the release mechanism and configured to regulate the dispersion of scented matter from the storage unit in accordance with the desires of a user. The control unit is an interface for the passenger to regulate scent dispersion. A sensor is also optionally included to monitor air quality from the ambient air. These and other unique features of the system and method are described herein below.

Referring now to the figures wherein like reference characters identify corresponding or similar elements in form and function throughout the several views. The following Figures describe system 99 and its associated features. With reference now to the drawings, an embodiment of the automatic car air freshener system are herein described. It should be noted that the articles “a”, “an”, and “the”, as used in this specification, include plural referents unless the content clearly dictates otherwise.

Referring now to FIG. 1 in the drawings, a side view of an automatic car air freshener system 101 in a vehicle 99 according to an embodiment of the present application is illustrated. Vehicle 99 is representative of any motorized transportation device and is not herein limited to the design shown. Vehicle 99 includes a dash 97 and an interior cabin environment 95. System 101 is configured to attach to vehicle 99 and selectively introduce the scented matter into existing air vents of vehicle 99. Controls are placed inside cabin 95 for a passenger to use. Other portions of system 101 are typically located within the engine compartment, however, this is not necessarily a requirement. System 101 may be partially or completely located within cabin 95. Cabin 95 is typically differentiated from the engine compartment along the firewall of vehicle 99.

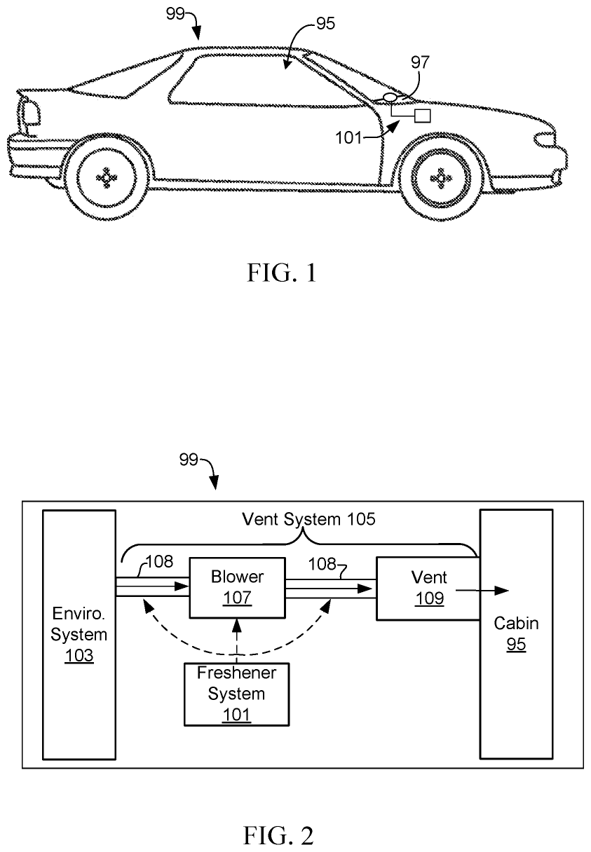

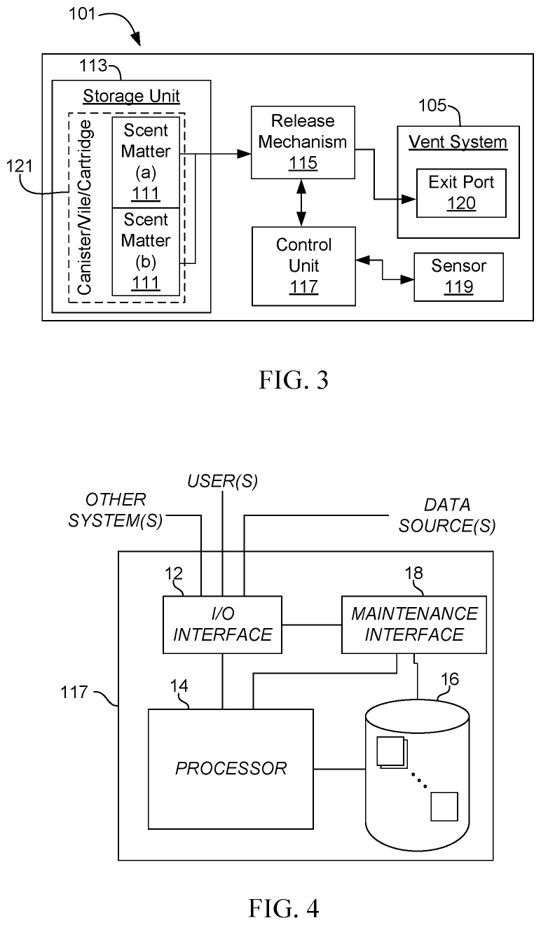

Referring now also to FIG. 2 in the drawings, a first schematic of the automatic car air freshener system 101 is illustrated. Within vehicle 99 is an environmental control system 103 that includes at least one of air conditioning and heating capabilities. System 103 is configured to force air (cold, warm, or vent from ambient outside air) into cabin 95 through vent system 105. Vent system 105 includes a blower 107, ducting 108, and one or more vents 109. System 101 of the present application is configured to disperse a scent matter 111 (see FIG. 3) into vent system 105 for distribution into the forced air flow feeding cabin 95.

System 101 is configured to integrate in various ways into vehicle 99. Scent matter 111 may be introduced in at least any one of three areas of vent system 105. First, scent matter may be distributed into the air flow of vent system 105 prior to the blower motor 107, wherein the blower motor 107 is used to draw in the scent and subsequently passing it through the vents 109 of the vehicle cabin 95. Secondly, scent matter 111 may be distributed directly into a portion of the blower motor 107 where the scent still passes within the blower 107 and then travels into cabin 95. Thirdly, scent matter 111 may be distributed into the air flow of vent system 105 after blower 107. In the first and third examples above, scent matter 111 is introduced directly into ducts 108. This ensures the ability to more adequately achieve a uniform dissemination of the scent matter within the vehicle cabin. Mechanism 115 dispenses scent matter 111 through an exit port 120.

Referring now also to FIG. 3 in the drawings, a schematic of system 101 is illustrated. System 101 includes a storage unit 113 containing a volume of scented matter 111, a release mechanism 115 in communication with storage unit 113, and a control unit 117 in communication with release mechanism 115 and configured to regulate the dispersion of scented matter 111 from the storage unit 113 in accordance with the desires of a user. The control unit 117 is an interface for the passenger/user to regulate scent dispersion. A sensor 119 is also optionally included and configured to monitor air quality from the ambient air in cabin 95.

Storage unit 113 is an interface coupled to a portion of vehicle 99 and serves as a housing unit for one or more canisters/cartridges 121. Each canister 121 may include one or more scented matters 111a/b. An example of canister 121 may be modeled after printer cartridges, may be a disposable vile, or may be a fillable container (i.e. styled similar to a windshield washer fluid container). Canister 121 are ideally removable and interchangeable to allow the user to swap out various scents. Each canister 121 is configured to couple to and be securely held by storage unit 113.

Scent matter 111 may come in various forms or states of matter. For example, scent matter 111 may be in the form of a liquid, solid, or gas within canister 121. System 101 is configured to disperse scented matter in a gaseous form. Ideally a vapor of scent matter 111 is dispersed into vent system 105. It is preferred that the mist have minimal moisture so as to avoid harmful effects of residue in system 105. A mist is not preferred. It is understood that scent matter 111 may also be an aerosol or be in a pressurized canister 121.

Release mechanism 115 is in fluid communication with canister 121 and scent matter 111. Upon instruction from control unit 117, mechanism 115 is activated so as to control the amount of scent matter 111 released. This in turn affects the potency of the scent matter in the cabin and frequency of dispersion.

Referring now also to FIG. 4 in the drawings, an exemplary schematic of control unit 117 is illustrated. Unit 117 is an interface operable by a passenger within vehicle 99 to regulate the dispersion of scent matter 111. Unit 117 may be controlled through direct contact with the user or may be regulated through software applications via an electronic device. This electronic device may be fully integrated into vehicle 99 or may be a portable personal electronic device of the user. Operational features/functions and information processed by unit 117 may be communicated with the user or any third party via any known communication methods. It is understood that unit 117 may be a simple mechanical or electromechanical device operable through manual control switches, knobs, and dials. FIG. 4 is used to illustrate unit 117 with the optional capabilities of a smart device, capable of monitoring, processing, and reacting to one or more inputs and data.

As a smart electronic device, unit 117 includes an input/output (I/O) interface 12, a processor 14, a database 16, and a maintenance interface 18. Alternative embodiments can combine or distribute the input/output (I/O) interface 12, processor 14, database 16, and maintenance interface 18 as desired. Embodiments of unit 117 can include one or more computers that include one or more processors and memories configured for performing tasks described herein below. This can include, for example, a computer having a central processing unit (CPU) and non-volatile memory that stores software instructions for instructing the CPU to perform at least some of the tasks described herein. This can also include, for example, two or more computers that are in communication via a computer network, where one or more of the computers includes a CPU and non-volatile memory, and one or more of the computer's non-volatile memory stores software instructions for instructing any of the CPU(s) to perform any of the tasks described herein. Thus, while the exemplary embodiment is described in terms of a discrete machine, it should be appreciated that this description is non-limiting, and that the present description applies equally to numerous other arrangements involving one or more machines performing tasks distributed in any way among the one or more machines. It should also be appreciated that such machines need not be dedicated to performing tasks described herein, but instead can be multi-purpose machines, for example smart phones, electronic tablets, and computer workstations, that are suitable for also performing other tasks. Furthermore the computers and machines may use transitory and non-transitory forms of computer-readable media. Non-transitory computer-readable media is to be interpreted to comprise all computer-readable media, with the sole exception of being a transitory, propagating signal.

The I/O interface 12 provides a communication link between external users, systems, and data sources and components of unit 117. The I/O interface 12 can be configured for allowing one or more users to input information to unit 117 via any known input device. Examples can include a keyboard, mouse, touch screen, microphone, and/or any other desired input device. The I/O interface 12 can be configured for allowing one or more users and devices to receive information output from unit 117 via any known output device. Examples can include a display monitor, a printer, a speaker, and/or any other desired output device. The I/O interface 12 can be configured for allowing other systems to communicate with unit 117. For example, the I/O interface 12 can allow one or more remote computers to access information, input information, and/or remotely instruct unit 117 to perform one or more of the tasks described herein. The I/O interface 12 can be configured for allowing communication with one or more remote data sources.

The database 16 provides persistent data storage for unit 117. While the term “database” is primarily used, a memory or other suitable data storage arrangement may provide the functionality of the database 16. In alternative embodiments, the database 16 can be integral to or separate from unit 117 and can operate on one or more computers. The database 16 preferably provides non-volatile data storage for any information suitable to support the operation of unit 117. Database 16 stores information.

The maintenance interface 18 is configured to allow users to maintain desired operation of unit 117. In some embodiments, the maintenance interface 18 can be configured to allow for reviewing and/or revising the data stored in the database 16 and/or performing any suitable administrative tasks commonly associated with database management. This can include, for example, updating database management software, revising security settings, and/or performing data backup operations. In some embodiments, the maintenance interface 18 can be configured to allow for maintenance of processor 14 and/or the I/O interface 12. This can include, for example, software updates and/or administrative tasks such as security management and/or adjustment of certain tolerance settings.

The processor 14 is configured to process any one of a number of functions and information, including at least some of environmental air quality in cabin 95 and operational performance of mechanism 115. Processor 14 may access and compare information stored on database 16. Processor 14 can include various combinations of one or more processors, memories, and software components. Additionally, processor 14 may be configured to run various software based applications to perform various tasks and store specific information for a user.

Unit 117 is configured to receive input from a user so as to regulate the timing of release of scented matter 111 from the storage unit 113. Furthermore control unit 117 is configured to receive input from a user so as to regulate the potency of scented matter released from the storage unit. A user may manually control each dispersion or wherein unit 117 is “smart”, it may automatically regulate it according to predefined preferences of the user. For example, dispersion of the scented matter may be made in accordance with one or more factors. Such factors may be any of at least the following: receipt of power to the control unit, a predefined interval pattern stored on the control unit, and in response to air quality readings within the ambient air.

As stated previously, system 101 may optionally include air sensor 119 configured to monitor and obtain air quality data from the ambient air. This air quality data is communicated to unit 117 and subsequently processed. An indicator (on unit 117 or an electronic device) may be provided to the user to take manual operational control of dispersions or unit 117 may automatically selectively perform one or more functions in view of the processed air quality data.

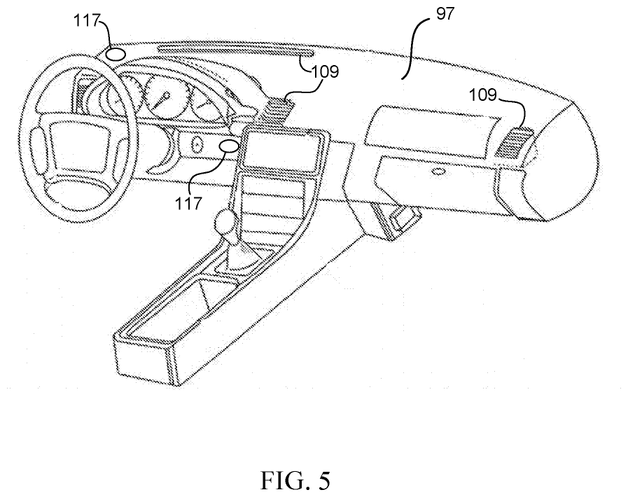

Referring now also to FIG. 5 in the drawings, an interior perspective view of vehicle 99, and in particular of cabin 95 is illustrated. Dash 97 is shown in more detail with unit 117 shown attached at different exemplary locations. Each location is not herein limiting. As stated before, other portions of system 101 may also be located in cabin 95. It is important to note that through unit 117, the user may control such dispersions of one or more scents. By relying on existing forced air systems, system 101 is simpler and more easily retrofitted into existing vehicles. System 101 may be integrated into vehicles at either a pre or post original equipment manufacturer purchase. For example, system 101 may be purchased aftermarket and applied to a used vehicle. When installed on a brand new OEM vehicle, an auto manufacturer may elect to provide system 101 as optional or standard equipment. System 101 may therefore be operably controlled through the vehicle's electronic/manual interface systems.

The current application has many advantages over the prior art including at least the following: (1) ability to regulate the amount of scent in the cabin; (2) ability to regulate the potency of scent; (3) optional self-regulating of dispersion based on monitored air quality data; (4) interchangeable canisters; and (5) even distribution of the scent matter via ducts of vehicle environmental control system.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

1. An air freshener system for a vehicle, comprising:
a storage unit containing a volume of a scented matter, the scented matter being contained within a removable canister, the canister is located within a cabin of the vehicle;
a release mechanism in communication with the storage unit, the release mechanism routing the scented matter into a vent system of the vehicle through an exit port, the exit port coupled to the vent system; and
a control unit in communication with the release mechanism and configured to regulate the dispersion of scented matter from the storage unit in accordance with the desires of a user, the control unit is located within the cabin of the vehicle and the storage unit is located in an engine compartment of the vehicle, the scented matter being dispersed into the vent system of the vehicle through the exit port in communication with the release mechanism.

2. The system of claim 1, wherein the scented matter is at least one of a liquid, a solid, and a gas.

3. The system of claim 1, wherein the scented matter is an aerosol.

4. The system of claim 1, wherein the control unit is operable by a user.

5. The system of claim 4, wherein the user is within a vehicle.

6. The system of claim 1, wherein the control unit is configured to receive input from a user so as to regulate the timing of release of scented matter from the storage unit.

7. The system of claim 1, wherein the control unit is configured to receive input from a user so as to regulate the potency of scented matter released from the storage unit.

8. The system of claim 1, wherein the control unit is configured to receive input from a user so as to regulate the timing of release of scented matter from the storage unit.

9. The system of claim 1, wherein the control unit is configured to automatically disperse scented matter according to one or more factors.

10. The system of claim 9, wherein the one or more factors is at least one of a receipt of power to the control unit, a predefined interval pattern stored on the control unit, and in response to air quality readings within the ambient air.

11. The system of claim 9, wherein the control unit further includes a sensor to obtain air quality data from the ambient air.

12. The system of claim 11, wherein the control unit is configured to process the air quality data and selectively regulate the dispersion of scented matter.

13. The system of claim 1, wherein the scented matter is distributed through the ambient air via a forced air system, the forced air system incorporated into the vent system.

14. (canceled)

15. The system of claim 1, wherein the storage unit includes a plurality of different scented matters, the control unit configured to selectively alternate between the different scented matters for dispersion.

16. The system of claim 15, wherein the user is able to selectively nullify a prior scented matter by introducing a subsequent scented matter by manipulating the control unit.

17. The system of claim 1, further comprising:
a portable electronic device independent of the control unit, the portable electronic device configured to communicate with the control unit to permit remote user control of scented matter dispersion functions.

18. The system of claim 1, wherein the scented matter is routed into the vent system prior to a blower motor within the vent system of the vehicle.

19. The system of claim 1, wherein the scented matter is routed into the vent system after a blower motor within the vent system of the vehicle.

20. The system of claim 1, wherein the scented matter is routed into the vent system directly into a blower motor within the vent system of the vehicle.

21. The system of claim 1, wherein the scented matter is routed through the vent system and is uniformly disseminated through each interior cabin vent in the vehicle.
